(19) United States
(12) Patent Application Publication
(10) Pub. No.: US 2013/0144184 A1
(43) Pub. Date: Jun. 6, 2013

(54) METHOD FOR SUPPRESSING THE LUMINANCE ARTIFACTS OF LCD MONITORS IN ELECTROPHYSIOLOGY OF VISION

(71) Applicant: Gualtiero Regini, Roma (IT)

(72) Inventor: Gualtiero Regini, Roma (IT)

(73) Assignee: INGENESI, Roma (IT)

(21) Appl. No.: 13/813,989

(22) PCT Filed: Jul. 28, 2011
(86) PCT No.: PCT/IT2011/000272

§ 371 Date: Feb. 4, 2013

(30) Foreign Application Priority Data
Aug. 4, 2010 (IT) RM2010A000439

(51) Int. Cl. A61B 3/00

(52) U.S. Cl. CPC A61B 3/0041
USPC 600/558

(57) ABSTRACT

A visual stimulation method and system for the field of electrophysiology of vision to obtain the suppression of the recording artifacts due to the luminance transients of the LCD monitors during the inversion of a main stimulation pattern (17) composed of a plurality of geometric elements, each of which is represented by a pair of complementary micropatterns (21, 22) and where the micropattern of each pair alternate between them in a synchronised manner with the frame rate.

### FIELD OF THE INVENTION

The present invention applies to the field of diagnostic tools for testing of visual functions and in particular for the recording of Visual Evoked Potentials (VEP) and pattern-electroretinogram (PERG).

### STATE OF THE ART

The diagnostic tools for diseases or for research in biological and cellular mechanisms of the human/animal visual system, since many years make use of photopic stimulators and relevant equipment for the recording of visual evoked potentials called VEP and pattern-electroretinogram called PERG.

For the performance of these electro-functional tests the subject is placed in front of various visualisation systems such as cathode ray tube (CRT) monitors, liquid crystal display (LCD), projectors, LCD glasses, or LED diode array, etc.

The appropriate electrodes are positioned on the specific locations of the subject depending upon the type of test. The stimulation pattern is therefore made to vary in time. The periodic changes in geometry, contrast and luminance of the stimulation pattern induce bio-electric potential changes in the subject at the retinal and cortical levels. These techniques are well known and recognised as part of medical practice and scientific research.

The most commonly and traditionally used stimulator consists of a normal CRT monitor, which displays the stimulation pattern such as checkerboard or grating, horizontal or vertical bars or polygons, where bright and dark luminous areas alternate cyclically. The inversion frequency of dark and light elements of the pattern typically ranges from fractions of a hertz up to tens of Hz.

This range of variations is upside or upward limited because the retina in the case of PERG, or the cerebrocortical zone in the event of the VEP, does not react to stimuli of higher frequencies. When sequences of images are projected at frequencies higher than 40-50 Hz then a temporal fusion effect is generated in the human visual system that produces a vision of a stable and flicker-free image. It is important to note that many types of stimulators, including CRTs and LCDs in fact, do not produce static images, instead the image is replicated many times per second, but the visual effect is that of a fixed image due to the above-mentioned temporal fusion effect.

The guidelines that outline the requirements for patterns stimulators and the instrumentation in general in the field of electrophysiology of vision are outlined in the following publications of ISCEV (International Society for Clinical Electrophysiology of Vision):

1. “Guidelines for calibration of stimulus and recording parameters used in the Clinical Electrophysiology of Vision” (Documenta Ophthalmologica 107: 185-193, 2003).

2. “ISCEV standard for clinical pattern electroretinography-2007 update”, Doc Ophthalmol (2007) 114:111-116

3. “ISCEV standard for clinical visual evoked potentials (2009 update)”, Doc Ophthalmol (2010) 120:111-119.

Among the requirements for VEP and PERG stimulators it is indicated that the monitor must not produce luminance transients at the time of the pattern reversal. While almost all the relevant scientific publications cited among the methods the usage of the CRT monitors, some of the most recent publications refer to experiments conducted with the use of LCD or plasma monitors. The introduction of these new technologies is viewed with caution and are subject to thorough evaluation and comparisons:
Abstracts of the XLV International Symposium of ISCEV-Hyderabad, India, 25-29 Aug. 2007 Oral paper 39, pages 47, 48; “Review of the ISCEV calibration guidelines”.
Abstracts: XLVII ISCEV International Symposium Abano Terme-Padova, Italy; Jul. 6-10, 2009, page 55; “Comparison of VEP results performed on CRT and LCD displays and evaluation of photometric calibration of the devices”

A technical analysis of the requirement to have PERG and VEP stimulation monitor without luminance transients can be conducted as follows.

A characteristic stimulation pattern in the form of checkerboard or grating with black and white squares is denominated A, and an inverse geometrical pattern is denominated B, i.e. where white elements of A correspond to the black elements of B and vice versa, it is clear that for any stimulation monitor the requirement of mean luminance of the stimulation pattern remaining constant over time is based on three key assumptions:
1) The pattern A and pattern B, which alternate periodically on the monitor screen, must have the same overall spatial mean luminance (measurable in cd/m²).
2) The monitor should not introduce variations in luminance perceptible to the eye, both when viewing the pattern A and pattern B. In other words, the luminance of these patterns must appear constant throughout the time of their display to the stimulated person.
3) When changing the pattern (reversal from A to B or from B to A) the monitor must not cause variations in mean luminance of the displayed pattern.

Requirement 1) is simply obtainable by a pattern that has an even number of elements of which half are blacks and the other half are whites. One can observe as in this case that the above-mentioned requirement depends upon the geometry of the pattern and is independent of the technology of the monitor displaying the pattern.

Requirement 2) is related to the technology of stimulator monitor as both the CRT monitors and the LCD are suitable to meet this requirement; both operate at a frequency or frame rate above 60 Hz because at these frequencies, as already mentioned, the temporal fusion effect is generated in the visual system that allows vision of a stable, flicker-free image. The resulting effect is then of an image without flicker (checkerboard A or checkerboard B) with constant luminance intensity.

Requirement 3) can be easily provided by the CRT-type monitor on the condition that the change of pattern from A to B and vice versa is synchronised with the vertical refresh rate. The CRT monitor with synchronised pattern reversal, has no pattern luminance transients perceptible to the retina because the screen is completely redrawn by the electron beam at each video frame. If the persistence of the phosphor is sufficiently short then the electron beam finds a “blank” screen at any retrace of the vertical synchronism and can then begin to draw both the pattern A and B without causing a change in the mean luminance of each frame. The resulting effect for the vision is therefore a flicker-free image (checkerboard A or checkerboard B) with constant luminosity.

As a principle the same does not happen, with the current active-matrix LCD monitors, including those with grey to grey response time (TGG) of less than 5 ms. These monitors typically operate with an update of one line at a time, starting for example from the top line until reaching the last line at the bottom of the screen. Each line of the screen consists of a certain number of pixels. Three elements that operate at different wavelengths of the visible typically red, green and blue normally correspond to each pixel in the colour LCD monitor. When updating a line, the turning on or off mechanisms of individual pixels lead to activation times ON (known as Tbw, black to white) and OFF (known as Twb, white to black), which are generally different. Even if the change of pattern is synchronized with the vertical synchronism of the frame, this asymmetry of times is the main cause of the variation of the instantaneous luminance during the moment of pattern reversal. This luminance transient occurs at each reversal of the pattern and is different from what is prescribed by the ISCEV protocol. Thus the said luminance transient can cause a more or less severe artifact in the recording of the PERG or VEP.

It is to be noted that this artifact is perfectly synchronized with the pattern reversal and for this reason, the examiner can not distinguish whether the corresponding recorded signal is a consequence of the variation of contrast, which should ideally be, either the luminance transient or both the phenomena.

In the last decade, the large-scale commercial availability of low cost and highly compact LCD have made CRT monitors obsolete both for television and computer applications, as well as in medical diagnostic imaging. Therefore, CRT monitors have become hard to find because they are obsolete.

Many patent applications have been published regarding the improvement of LCD monitors technology in general, to obtain continuously faster response times for example using the technique of “overdrive” and etc. One example is represented by the patent applications WO2010039576, US 2010165222, and JP20000254279, however efforts to reduce the overall time TGG mitigate but not solve the problem of asymmetry Tbw-Twb. It is likely that in the near future, the widespread diffusion of Active-matrix organic light-emitting diode or AMOLED panels technology may instead effectively solve the aforementioned defect.

The problem remains that the current commercial LCD monitors, while having the advantages of compactness, portability and affordability, present limits and problems due to the presence of this luminance transient during the pattern reversal for diagnostic applications such as VEP, PERG and derivatives.

Because of this flaw, the performance of LCD monitors used as photopic stimulators are not ideal, but the near obsolescence of CRT monitors leads the market for this diagnostic equipment to adopt the technology of the LCD pattern stimulators.

An example of use of LCD monitor for VEP is described by the Australian patent application No. AU2007200577 A1 discloses a system for recording the visual electrophysiological response of a subject to a pattern stimulation, comprising an LCD monitor showing a pattern and providing a photopic stimulation of a subject, electrodes applied to the subject, a preamplifier of signal from said electrodes, an acquisition and control system, a monitor for an operator and a pattern generator VPG.

### SUMMARY OF THE INVENTION

The purpose of the present invention is to overcome the disadvantage of the aforementioned luminance transient typical of these LCD monitors, providing a method and a system capable of recording Visual Evoked Potentials and pattern-electroretinogram by using commercial LCD monitors with function of stimulator and eliminating artefacts caused by luminance transient in compliance with ISCEV standards.

By way of a non-limiting example, the two aforementioned checkerboard patterns A and B, but with the possibility of using alternative patterns such as rectangles or polygons, consisting of whites and blacks elements, the elimination of the luminance artifact has been achieved according to the following criteria.

The luminance transient of the LCD monitor is manifested at each pattern reversal, from A to B and from B to A. Namely, Tp is the period of pattern reversal and Fp=1/Tp is the pattern reversal frequency. For the foregoing reasons, the luminance transient Tt has a period equal to the reversal of the pattern (Tt=Tp) or in terms of frequency Ft=Fp.

The present invention does not eliminate the luminance transient as such but acts upon the negative effect of this transition. This is achieved by changing the frequency of repetition of the luminance transient Ft in order to separate it from the frequency of reversal of the pattern Fp and simultaneously increasing the frequency Ft beyond the frequency of visual temporal fusion, so that the frequency of repetition of luminance transition Ft is so high that it does not cause artifacts during the recording of PERG and VEP attributable to luminance transient.

In particular, with the present invention the luminance transient is replicated with the same frame rate frequency Fq of the LCD monitor. It follows that a luminance pulse caused by this transient has a much higher frequency Ft=Fq than the frequency Fp of the pattern reversal. Selecting a LCD monitor having frame rate frequency Fq beyond the limits of visual fusion (e.g. monitor with 60 Hz or higher frequency), then the resulting luminance pulse will be perceived as a constant luminance.

The method of this invention achieves this result by breaking up the stimulation pattern, from now on referred to as the main, made up of geometric structures such as squares, rectangles, or polygons, consisting of grey or white elements or any other colour, arranged in a proper sequence of pairs of secondary patterns, henceforth referred to as micropatterns, which alternate with each other in a synchronized manner with the frame rate, as in the following.

These pairs of micropatterns and the related temporal sequence, are generated by the present invention in its preferential illustrative implementation, described in detail later.

### BRIEF DESCRIPTION OF THE DRAWINGS

FIG. 1 shows schematically the recording system and the subject to be examined.

FIG. 2 illustrates the main patterns.

FIGS. 2a and 2b illustrate micropattern pair corresponding to the checkerboard.

FIG. 3 illustrates the main pattern entitled “dartboard”.

FIG. 3a shows the main pattern with the form of vertical bars.

FIG. 3b shows a pair of complementary micropattern of horizontal lines.

FIG. 4 illustrates a stimulation pattern with mask.

FIG. 5 shows the luminance transient of a LCD monitor operated according to the state of the art.

FIG. 6 illustrates the luminance transient of a LCD monitor operated according to the present invention.

FIG. 7 shows the block diagram of the pattern generator VPG in its preferential implementation.

FIG. 8 shows the flowchart of Micropattern Sequencer.

### DESCRIPTION OF THE PREFERRED EMBODIMENT

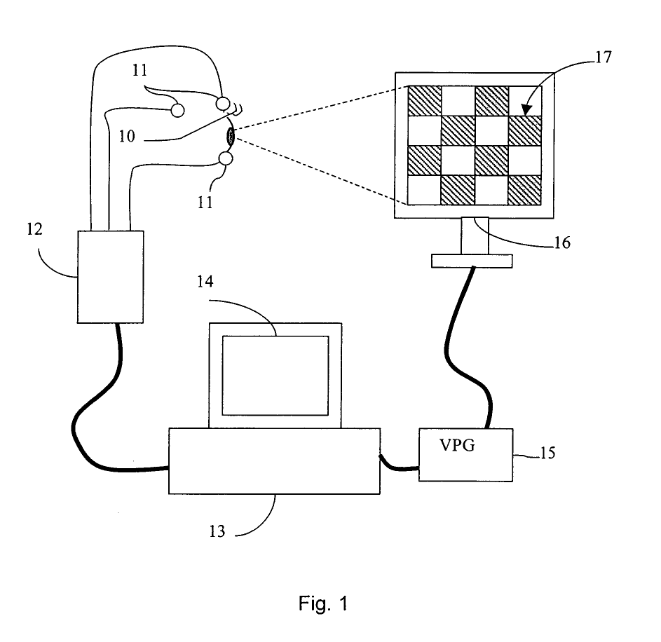

The system according to the present invention is shown in FIG. 1. The system consists of a commercially available 19-inch LCD monitor 16 commonly used with personal computers, which displays the pattern 17 and provides the photopic stimulation of the subject 10. The LCD monitor 16, for example, operates at the vertical frequency range of 50-150 Hz and typically at 64 Hz, has a typical response time of 5 ms TGG along with a transition time Tbw typically of 6 ms, has a typical transition time Twb of 2 ms and has a resolution e.g. of 1280×1024 pixels RGB. The maximum luminance of the monitor e.g. is 300 cd/m², while the mean luminance level required for the stimulation in this example is equal to 40 cd/m². The system in FIG. 1 is also composed of electrodes 11 applied to the subject 10 for the detection of bio-electric signals induced by the aforementioned stimulation, a signal preamplifier 12 of these electrodes, a control and acquisition system 13, which digitises and processes the amplified signal from the latter preamplifier, a monitor 14 for the operator, a pattern generator 15, hereafter called VPG (Video Pattern Generator) that drives the LCD monitor 16 as a stimulator.

During PERG or VEP examination the subject 10 is positioned in front of the LCD monitor 16 as a stimulator which displays the pattern 17 for the structured photopic stimulation, in the present example in the form of checkerboard with a frequency of 16 reversals per second. One or more pairs of electrodes 11 are applied to the eye of the subject 10, of which one is an active electrode and one is reference electrode, in addition to a ground electrode; the signal picked up by the active and reference electrodes is amplified by the amplifier 12 in differential mode and is sent to the acquisition and control system 13 for presentation on the monitor 14 for the operator. The operator provides for the setting, implementation, and processing of electrofunctional test data through the aforementioned acquisition and control system 13. Finally, the LCD monitor 16 as a stimulator is controlled by the pattern generator VPG 15.

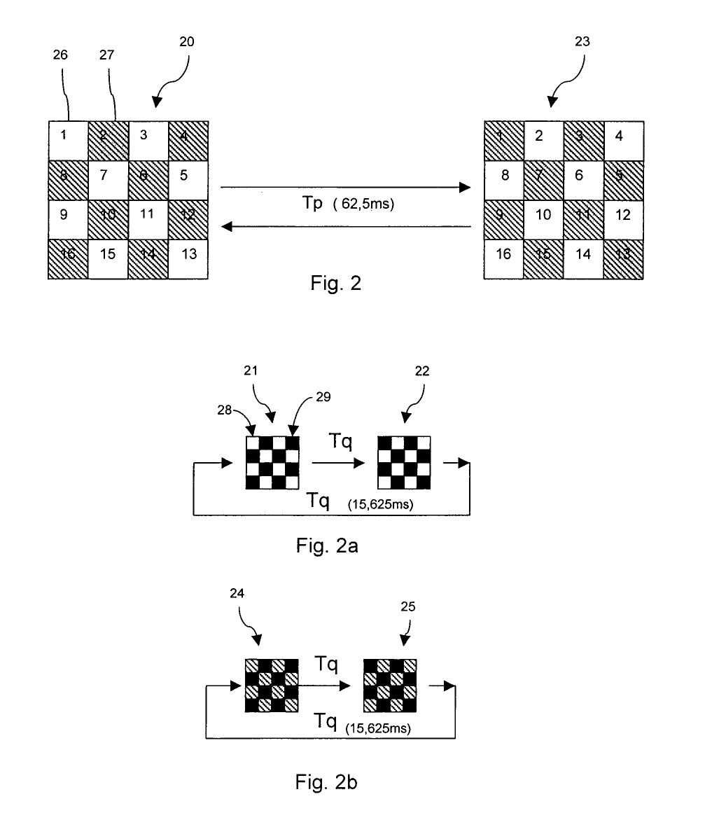

With reference to FIG. 2, one of the patterns that can be reproduced by abovementioned VPG generator 15 is represented as non-limiting example, where the main pattern has the form of checkerboard 20 in this case. It is constituted by an equal number of square elements, the half of which are white 26 and the remaining half are grey 27. In a consecutive temporal phase of visual stimulation, the main pattern 20 is reversed and it assumes the aspect 23. As an example, the reversal cycle of the main pattern repeats every 62.5 ms. The elements of the main pattern 20 or 23 are numbered 1 through 16 to decisively identify the position of each element within the main pattern. It's obvious that the white and grey colours are mentioned here as examples only and may be replaced by any other colour available in the RGB chromatic triangle of the specific monitor.

The above-mentioned function is well-known in the art, while the specificity of the VPG generator 15 of this invention is that the elements 26 and 27 of the main stimulation patterns are represented by pairs of micropatterns shown in an enlarged view in FIG. 2a and FIG. 2b, where the first pair 21 and 22 corresponds to white elements 26, the second pair 24 and 25 represents the grey elements 27 and where the micropatterns of each pair mutually alternate in a synchronized manner with the frame rate, which in this example is of 64 Hz and hence with a period Tq=15.625 ms.

The VPG generator 15 of this invention has the characteristic of generating pairs of micropatterns henceforth referred to as “complementary” or “complementary geometry” such as the pair 21, 22, where these micropatterns are composed of a plurality of luminous geometric elements 28 and black geometric elements 29.

The pair of complementary geometry micropatterns, produced by the VPG generator 15 has the following three general properties, which are outlined here solely as a non-limiting example, referring to the pair of micropatterns 21, 22 in FIG. 2a: Each micropattern of the pair of micropatterns 21, 22 is shaped from an even number of elements the half of which are black 29 and the remaining half are luminous 28 in order to display the same mean luminance when the micropatterns of the pair of micropatterns 21 and 22 mutually alternate.

The luminous elements 28 related to the pair of micropatterns 21, 22 have the same colour of the element 26 of the represented main pattern, but have a double luminous intensity, so as to display the same mean luminance of the element 26 of the main pattern.

For each pair of micropatterns 21, 22, the luminous elements of the first micropattern correspond to the black elements of the second micropattern and vice versa, so that each element remains luminous for a single video frame and in the next frame is turned off and assumes black colour. This feature allows generation of a luminance transient at each video frame.

In order that the pair of micropatterns 21, 22 is not noticeable to the eye of the subject 10, it is desirable that the micropatterns elements size is as small as possible. Thus another feature of the VPG generator 15 is to generate micropatterns, whose elements coincide with the pixel of the LCD monitor 16, in its highest native resolution, where pixel means collection of three RGB elements of the LCD adapted to the generation of the colour spectrum.

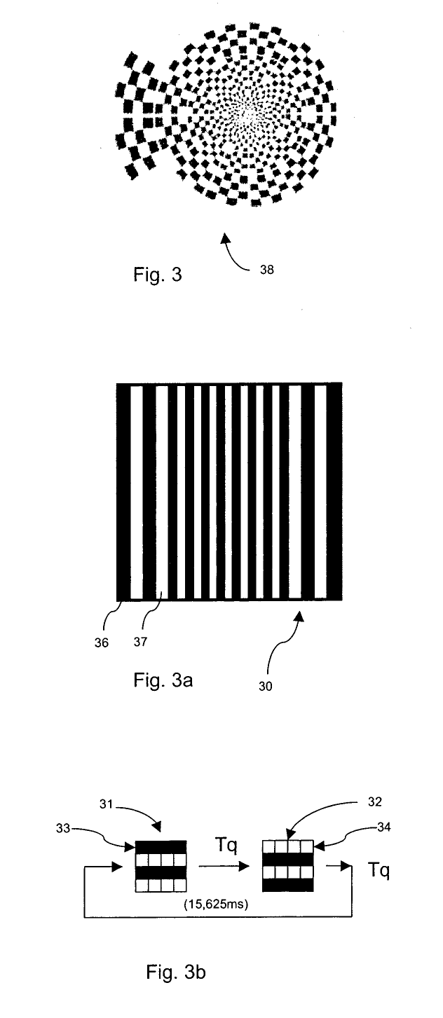

It is evident that there are many variants of the pair of micropatterns 21, 22 of FIG. 2a that meet the above-mentioned three properties, such as the pair 31, 32 of FIG. 3b, that may be used to represent both the white element 37 and the grey element 36 of the main pattern 30. Especially the geometry of alternating lines, luminous 34 and black 33 of the micropatterns 31 and 32, where individual elements correspond to the same pixel of the LCD monitor 16, has the advantage of requiring less bandwidth by the video VPG generator 15 and therefore its implementation is easier and less expensive.

That said, the VPG generator 15, through the main patterns 20 and 23 and the pairs of micropatterns 21, 22 and 24, 25 will apparently produce the same images of a conventional stimulation patterns generator that only displays the patterns 20 and 23. The reason for this effect is due both to the high switching frequency of pairs of micropatterns that the eye perceives to be constantly present, and the complementary geometry that produces the effect of a surface of uniform colour and intensity.

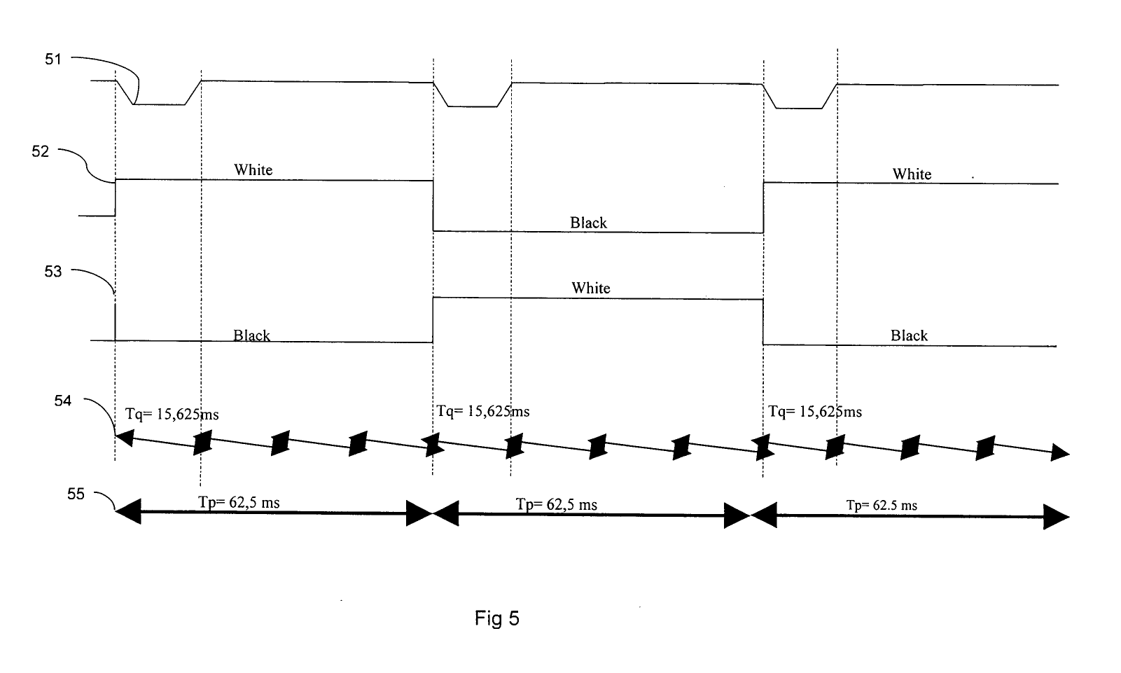

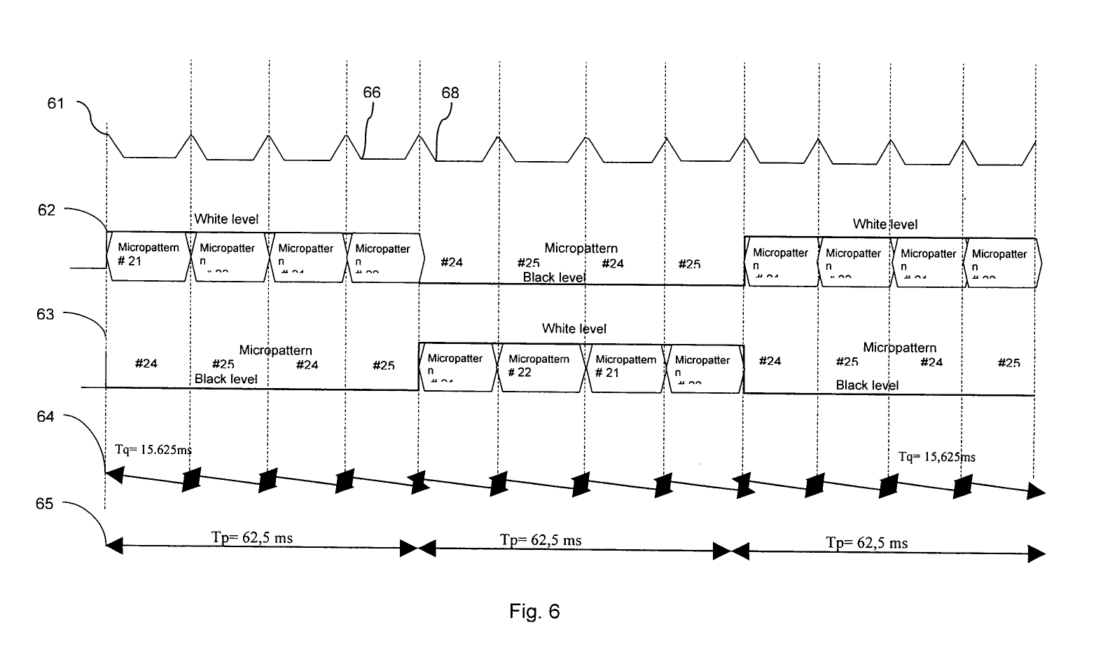

To understand the temporal sequences of the stimulation images generation according to the present implementation of the VPG 15 generator, reference is made to the waveforms illustrated in FIG. 5 and FIG. 6.

The FIG. 5 depicts the hypothetical waveform of the luminance transient of LCD 16 monitor as a stimulator, controlled according to the state of the art, when the displayed checkerboard patterns 20 or 23 mutually alternate without the use of the micropatterns. For ease of exposition, it is hypothesised in the present explanation that the elements 27 of the main pattern are of black colour instead of grey.

The trace 52 represents the colour of the odd elements as a function of time, where the high level corresponds to white and the low level corresponds to black.

Similarly the trace 53 represents the colour of the even elements as function of time. The trace 51 represents the luminance and the hypothetical luminance transients of LCD 16 monitor as a stimulator when it is controlled according to the current state of the art. The luminance transient takes place after each main pattern reversal, with period Tp=62.5 ms as shown in the specific example of the trace 55. The period of the video frame 54 is 15.625 ms. Typically, the period during which the peak luminance transient occurs it is the first frame after the pattern reversal, in the LCD monitors with response time of less than 5 ms. Bear in mind that the luminance transient 51 is due to the asymmetry of the dynamics of the turning on/off of the pixels, i.e. the times Tbw and Twb of the LCD 16 monitor and that this transient does not conform to the technical requirements of the ISCEV standards.

The FIG. 6 depicts the waveform of luminance transient of the LCD 16 monitor 16 as a stimulator controlled by the device and method of this invention, where the VPG generator 15 produces alternating checkerboard patterns 20 and 23 and the related pair of micropatterns 21, 22 in FIG. 2a and the micropatterns 24, 25 in FIG. 2b.

For ease of exposition, it is hypothesised in the present explanation that the elements 27 of the main pattern are of black colour instead of grey, therefore in this case the micropatterns 24, 25 have all the elements of black colour.

The trace 62 represents the colour of the odd elements as a function of time, where the high level corresponds to the white and the low level corresponds to the black. The white level is obtained by means of micropatterns 21 and 22, which mutually alternate with each period of the frame 64 equal to 15,625 ms.

The colour of the micropatterns 24 and 25, in this case both fully black, is constantly equivalent to black.

Same as aforementioned trace 62, the trace 63 represents the colour of the even elements as function of time. The trace 65 indicates the period of main patterns 20 and 23 reversal.

As already mentioned, the subject 10 would not notice differences between this stimulation micropattern image and the one generated according to the state of the art without micropattern in FIG. 5, apart from the mean luminance of 51 and 61 of the two methods. This difference in mean luminance can also be easily corrected through an instrumental calibration of luminous intensity of the micropatterns. As an example, to obtain the mean luminance of the main patterns 20, 23 equal to 40 cd/m², the micropatterns 21 and 22 must have the white pixels of luminance equal to 160 cd/m² except for the aforementioned correction, as it can be easily deduced from the geometries of the checkerboard patterns 20, 21 and 22.

Despite the above-mentioned similarity of the displayed images, the method of the present invention involves the fundamental advantage that at each frame-sync a specially designed luminance transient is generated as shown in the trace 61. The trace 61 of the instantaneous luminance is then formed both by transients Trp(t) 68, due to main pattern reversal and by the transients Trmp(t) 66, caused by the pair of micropatterns 21, 22 reversal.

Now one can verify if the LCD 16 monitor has such a response that Twb<Tq and Tbw<Tq, the two types of transients are equivalent and have the same intensity and duration. In this case, all the luminance transients of the trace 61 are mutually identical. The luminance 61, varying periodically with frequency equal to the frame rate and the latter being higher than the visual fusion, will be perceived as a constant luminance that will add to the mean value of the main pattern, or else subtract in case of the transient with instant reduction of luminance as represented by the trace 61 of the present example.

The demonstration that luminance transients 66 and 68 are identical is based on the simple observation that in both the cases there are N white elements of the current micropatterns that become black, and as many N black elements of the current micropatterns that turn white, without influencing the overall luminance effects, where the switching elements are positioned in the space.

However it is important to note that the switching elements should have reached a condition of steady-state luminance, whether white or black during the time frame Tq, hence the above-mentioned requirement:

Twb<Tq and Tbw<Tq.

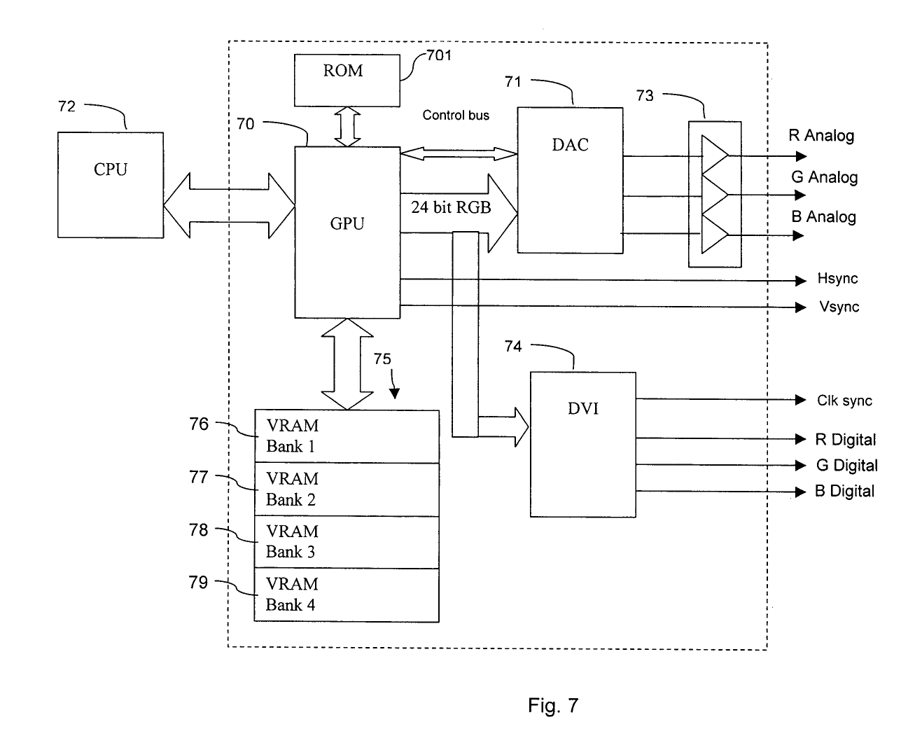

The FIG. 7 illustrates the VPG generator 15 of patterns in the present preferential implementation. By way of a non-limiting example, the VPG generator 15 consists of the following blocks.

A graphics processing unit (GPU) 70 provides the video sync generation and management of 24-bit RGB binary images, this GPU 70 is interfaced to a ROM 701 memory and is controlled by an external CPU 72 for setting the parameters of aspect and frequency of the main pattern.

The triple RGB DAC 71 converter receives digital input signals of the GPU, converts them into three RGB analogue signals with standard voltage levels between 0 V and 0.7 V and then follows a buffer interface 73 for connection to an external LCD monitor. There is also a DVI interface 74, which receives 24-bit RGB input data and provides the serialisation of R, G, B data and the clock sync data for interfacing with the latest LCD monitors with digital input.

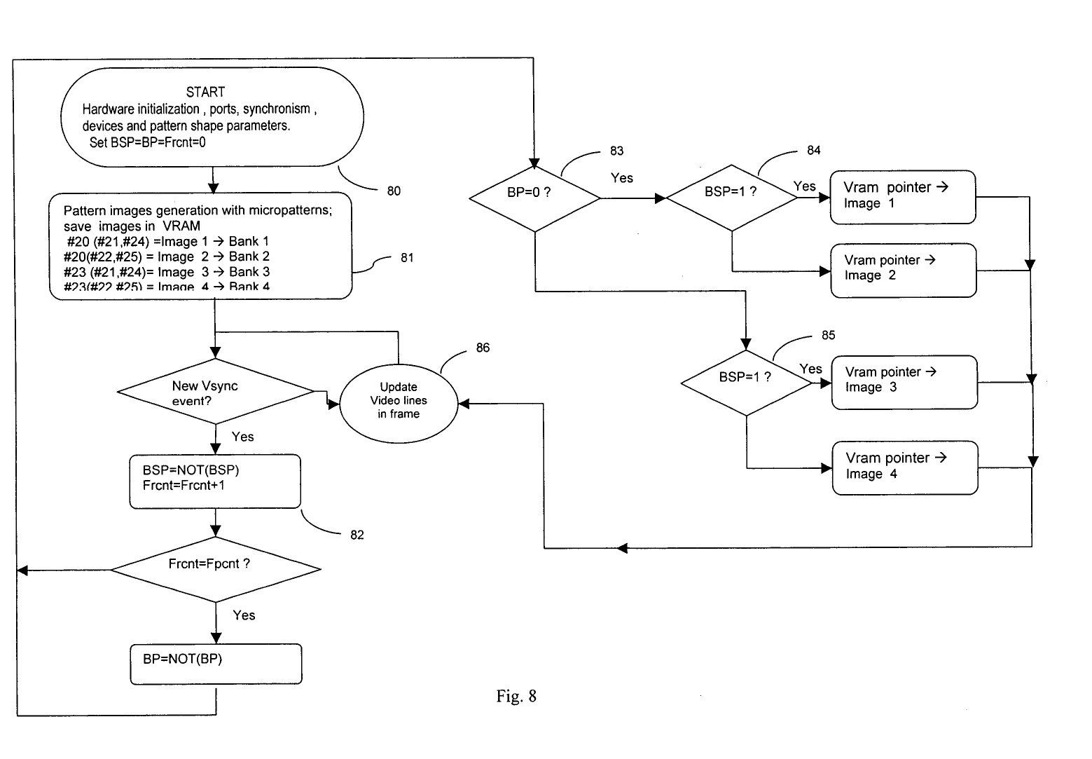

Another feature of the VPG 15 generator is the VRAM 75 video memory architecture, divided into four virtual banks with a data bus of 24-bits per pixel RGB that is optimised for the generation of complementary micropattern sequences according to the Micropattern Sequencer algorithm in FIG. 8, encoded within the ROM 701.

By way of a non-limiting example, each of these banks has size of 1280×1024×24 bits, adequate for containing an entire image of the LCD 16 monitor in its highest native resolution.

In particular the architecture of the VRAM 75 video memory in the specific example, contains the bitmap configurations of the main patterns 20 and 23 and of the pairs of micropatterns 21, 22 and 24, 25 as specified below:

Bank 1 (76): Bitmap Image No. 1 of the main pattern 20, where the white elements are constituted by micropattern 21 and grey elements of the micropattern 24;

Bank 2 (77): Bitmap Image No. 2 of the main pattern 20, where the white elements are constituted by micropattern 22 and grey elements of the micropattern 25;

Bank 3 (78): Bitmap Image No. 3 of the main pattern 23, where the white elements are constituted by micropattern 21 and grey elements of the micropattern 24;

Bank 4 (79): Bitmap Image No. 4 of the main pattern 23, where the white elements are constituted by micropattern 22 and grey elements of the micropattern 25.

The image that is sent to the monitor is picked up at each vertical sync of frame, from one of these banks, according to the Micropattern Sequencer algorithm of FIG. 8 and encoded in the ROM 701.

According to a different embodiment, given that the current personal computers are equipped with XGA-UVGA graphics cards that support the 3D graphics programming languages such as OpenGL 1.1 or later versions, or else Direct X 9 and later versions, the VPG generator 15 of patterns can be realised by means of this SVGA or UVGA video card preferably built into the computer control and acquisition system 13 and programmed to perform the Micropattern Sequencer algorithm of FIG. 8, encoded according to the syntax and rules of this graphic language.

FIG. 8 describes the Micropattern Sequencer algorithm, relevant to the control of the aforementioned VPG generator 15 in accordance with the temporal sequence shown in FIG. 6.

In particular there is an initialisation block 80, which provides for the configuration of the hardware of the VPG generator 15 and for the definition of parameters of externally pre-selected forms, such as height, width of checkers, inversion period.

The block 81 provides for the generation and loading of the four bitmap images in the corresponding four banks of VRAM 75 video memory, bank 1, 2, 3, 4, according to the combinations of patterns and micropatterns shown in the same block 81.

In block 82, at each sync frame, the bit called BSP is reversed, which will be used for determining the micropattern to be displayed; in same block 82, the frame sync counter called Frcnt is increased. In the control that follows, if the Frcnt equals the number Fpcnt, the bit designated BP is reversed, where Fpcnt is the counter of the periods of frame provided for the main pattern reversal. In the block 83 the control of bit BP takes place; in the blocks 84, 85 the control of bit BSP occurs.

The four possible combinations resulting from these controls 83, 84 and 85, determine the image to be displayed at the beginning of the current frame, i.e. the pointer to the VRAM 75 video memory bank containing the image to be displayed. In the next block 86 the VPG generator 15 hardware provides for the video frame update from the first line to the last line up to the next vertical sync frame. At the next Vsync (frame sync), the cycle described above has a new beginning.

The FIGS. 3 and 3a show two possible variants of shape of the main pattern of FIG. 2, like the dartboard 38 and the pattern of vertical bars 30. Said main patterns 30 and 38 represent just one example among the forms of visual stimulation that the VPG generator 15 can generate, not intending that the present invention is limited only to the use of the forms depicted.

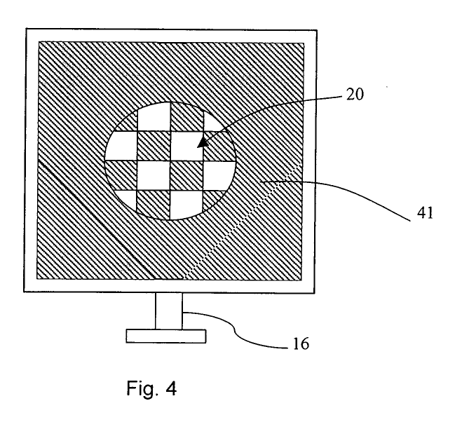

In the FIG. 4 a useful variation of the main pattern 20 of stimulation is illustrated, displayed on the LCD 16 monitor. In this variant there is an additional mask 41 which delimits the useful area of the main pattern 20 to a central portion of the LCD 16 monitor, in this example of circular shape with an opening comprised between 3 cm and 25 cm. This mask 41 ensures a constant illumination of the paracentral and peripheral areas of retina, excluding them from the stimulation, as in the case of the foveal VEP. The average luminance of the mask is typically equal to that of the variable main pattern 20, which in this example is 40 cd/m².

1-10. (canceled)

11. A method for obtaining the suppression of the luminance artifacts of LCD monitors used as visual stimulator for recording Visual Evoked Potentials (VEP) and pattern-electroretinogram (PERG) by means of an LCD monitor and a stimulation pattern, comprising:
generation and visualisation of a sequence of main patterns made of a plurality of elements, said sequence of main patterns being able to elicit the visual electrophysiological response in a subject, and
recordation of the visual electrophysiological response of the subject to said sequence of main patterns, said method being characterised in that each element is represented by means of a pair of complementary micropatterns having the following properties:
i. each micropattern of the pair is shaped from an even number of elements comprising equal pluralities of luminous and black geometric elements in order to display the same mean luminance when the micropatterns of the pair of complementary micropatterns alternate, in a synchronized manner, with the frame rate;
ii. the luminous geometric elements of the pair of micropatterns have the same colour of the represented element of the main pattern, but have double the luminous intensity, so as to display the same mean luminance of the element of the main pattern;
iii. when the pair of complementary micropatterns alternates, the luminous geometric elements of one micropattern replace the corresponding black geometric elements of the other micropattern and vice versa, so that each micropattern geometric element remains luminous for a single video frame and in the next frame is turned off and assumes the black colour.

12. The method according to claim 11, wherein equal pluralities of luminous geometric elements and black geometric elements are mutually spatially alternated square elements so that said pair of complementary micropatterns appear as a checkerboard.

13. The method according to claim 11, wherein said equal pluralities of black geometric elements and luminous geometric elements are mutually spatially alternated aligned elements so that said pair of complementary micropatterns appear as a composition of horizontal or vertical lines.

14. The method according to claim 11, wherein the geometric elements of complementary micropatterns coincide with the same pixels of the LCD monitor in its highest native resolution.

15. The method according to claim 11, wherein the frame rate is between 50 Hz and 150 Hz.

16. The method according to claim 11, wherein in addition a mask is displayed that narrows the visible area of the main pattern.

17. The method according to claim 11, wherein said mask has a width between 3 cm and 25 cm.

18. A system for recording Visual Evoked Potentials (VEP) and pattern-electroretinogram (PERG), of a subject by means of an LCD monitor, a stimulation pattern providing a visual stimulation of a subject, electrodes applied to the subject, a preamplifier of signals from said electrodes, an acquisition and control system, a monitor for an operator and a pattern generator VPG based on a PC graphics card supporting a 3D graphics programming language, characterised in that said PC graphics card is programmed to generate and display complementary micropatterns according to the method of claim 11.

19. The system according to claim 18, wherein the pattern generator VPG comprises a graphics processing (GPU), which is interfaced to a ROM memory and controlled by an external CPU, a triple RGB DAC converter that receives digital input signals of the GPU, followed by a buffer interface for the connection to LCD monitors, a video memory architecture (VRAM) said VPG being programmed to generate and display said complementary micropatterns.
